(19) United States
(12) Patent Application Publication
(10) Pub. No.: US 2023/0077072 A1
(43) Pub. Date: Mar. 9, 2023

(54) GRAPE SEED EXTRACTS FOR USE IN THE PREVENTION OR REDUCTION OF STRESS

(71) Applicant: Indena S.P.A., Milano (IT)

(72) Inventors: Antonella Riva, Milano (IT); Massimo Ronchi, Milano (IT); Giovanna Petrangolini, Milano (IT); Paolo Morazzoni, Milano (IT)

(73) Assignee: Indena S.P.A., Milano (IT)

(21) Appl. No.: 17/758,701

(22) PCT Filed: Jan. 14, 2021
(86) PCT No.: PCT/IB2021/050238

§ 371 Date: Jul. 13, 2022

(30) Foreign Application Priority Data
Jan. 15, 2020 (IT) 102020000000634

(51) Int. Cl. A61K 36/87; A61K 31/353; A61P 25/22; A61K 9/20; A61K 9/28

(52) U.S. Cl. CPC A61K 36/87; A61K 31/353; A61P 25/22; A61K 9/2009; A61K 9/2054; A61K 9/2866; A61K 9/2013; A61K 9/2027; A61K 9/2813; A61K 9/286; A61K 9/282

(57) ABSTRACT

Disclosed are compositions comprising grape seed extracts, in particular extracts with a proanthocyanidin content exceeding 95% by weight and a catechin and epicatechin content ≥5% and ≤15% by weight, which are useful to prevent or reduce the perception of stress symptoms, in particular the perception of stress among the male population.

### FIELD OF INVENTION

The present invention relates to pharmaceutical or nutraceutical compositions based on active plant ingredients for the prevention or reduction of stress.

### BACKGROUND TO THE INVENTION

Commonly, stress can be defined as a general syndrome of adaptation of an organism designed to restore a new inner balance following external factors, known as “stressors”. Stress can involve alterations in the inner balance of an organism at endocrine, mood, organic and/or biological level.

In general, a type of stress involving physiological reactions and alterations is defined as eustress (good stress). Conversely, a type of stress involving exaggerated reactions is defined as distress (bad stress); such stress can cause illnesses, which may be chronic, such as anxiety and depression, or psychosomatic disorders, which adversely affect the sufferer's quality of life.

Disorders caused by stress are usually treated with psychological therapies, or with diet supplements based on natural products or synthetic drugs.

Although the prior art provides several, sometimes controversial, definitions of stress, in the present invention the term “stress” is used to mean a pathological dysfunction caused by abnormalities in the autonomic nervous system; said system, interconnected with the central and peripheral nervous system, is responsible for maintaining the human body in stationary conditions, by modulating the activation of the sympathetic and parasympathetic nervous systems. Consequently, alterations in the operation of the autonomic nervous system, such as stress conditions, affect the central nervous system and the endocrine system, with repercussions on the immune response; moreover, stress leads to malfunction of the sympathetic and parasympathetic nervous systems, with consequent changes in heart movements. Said stress-generated alterations cause a number of other physical illnesses, such as cardiovascular and cerebrovascular disease, and mental disorders (International Journal of Applied Engineering Research 2018, vol. 13, no. 2, pp. 1460-1464). The natural active ingredients useful in the treatment of forms of anxiety include grape seed extracts, which are already known and used in the prevention and treatment of disorders of the cardiovascular system (RU2315616 C filed by G. V KURANOV; Belcaro G. et al. Evidence Based Complementary and Alternative Medicine, 2013, Art. ID 313142).

For example, CN108125013A (filed by DALIAN DENTBIO TECH CO LTD) describes a chewing gum containing various ingredients, including a grape seed extract, useful to alleviate (inter alia) the symptoms of anxiety and insomnia.

CN105918864A (filed by HEFEI ZHUGUANG GRAIN AND OIL TRADE CO LTD) discloses a composition based on fermented rice comprising various ingredients of plant origin, including a small proportion of grape seed extract, which is useful for various purposes, including improvement of the cognitive functions, improvement of inner spiritual energy (“tonifying qi”), and blood pressure reduction.

CN108936151A (filed by UNIV TIANJIN COMMERCE) discloses a composition based on Lycium ruthenicum (black Goji berries), and also containing various other plant-based ingredients, including a grape seed extract for the reduction of anxiety and treatment of insomnia.

WO2016020853A (filed by Rottapharm Biotech Srl) discloses a composition based on a purified cocoa extract and a purified grape seed extract for the treatment of various pathological conditions, including depressive syndromes and states of mental frailty/weakness. The grape seed extract contains between 16 and 25% by weight of flavan-3-ols, and catechins+epicatechins exceeding 15% by weight.

WO2016020855A (filed by Rottapharm Biotech Srl) discloses a grape seed extract for the treatment of various pathological states, including depressive syndromes, a process for the preparation thereof, and phaii iaceutical compositions containing said extract.

WO2019046660A (filed by TALBOTT Shawn) discloses a diet supplement containing natural extracts (combinations of pine-bark, grape-seed and apple extracts, comprising both fruit and peel) and prebiotics, which is effective in reducing bad moods.

Terauchi M. at al., Menopause: The Journal of The North American Menopause Society, 2014, Vol. 21, No. 9, pp. 990-996, reports on a study conducted on pre- and post-menopausal women to evaluate the effects of administration of an extract of proanthocyanidins from grape seeds, in particular an extract containing 85% proanthocyanidins (Gravinol™) at the dose of 100 mg/day or 200 mg/day. It emerged from said study that administration of the extract not only led to an improvement in the physical and psychological symptoms characteristic of the menopause, an increase in muscle mass and a reduction in blood pressure, but also reduced anxiety according to the criteria of the HADS questionnaire (Zigmond A S, et al. Acta Phychyatr. Scand. 1983; 67; 14: 117-126).

Vogels N., European Journal of Clinical Nutrition, (2004) 58, pp. 667-673, reports on a study conducted to evaluate the effects of administering 300 mg/day of a grape seed extract containing over 90% proanthocyanidins on food intake in humans. The author comments that no changes in mood were observed following administration of the extract (p. 670, right-hand column, lines 14-15 from top of page: “ . . . mood and tolerance were not affected when using the grape-seed supplements”).

Alrefaie Z., Int. J. Vitamin. Nutr. Res., 85 (5-6), 2015, 282-291, reports on a study conducted to evaluate the anxiolytic effect of a grape seed extract in hypercholesterolaemic rats. The extract was obtained by drying the seeds for 72 h at 70° C., followed by extraction with petroleum ether.

However, none of said documents describes the specific use of grape seed extracts for the prevention or reduction of stress.

### DESCRIPTION OF THE INVENTION

The Applicant has now discovered that grape seed extracts, in particular extracts having a proanthocyanidin content exceeding 95% by weight and a catechin and epicatechin content ≥5% and ≤15% by weight, can be advantageously used to prevent or reduce the perception of stress symptoms, in particular the perception of stress symptoms among the male population.

“Perception of stress symptoms” here means one or more of the feelings measured in an individual using the “Perceived Stress Questionnaire” (PSQ20) (Fliege et al. Psychosom Med. 2005 January-February; 67(1):78-88), which evaluates the feelings of worry, tension, joy and demands experienced by an individual. Each feeling is evaluated with five questions.

Thus in a first aspect thereof, the invention relates to grape seed extracts for use in preventing or reducing the perception of stress symptoms, in particular among the male population.

In a second aspect thereof, the invention relates to grape seed extracts for use in preventing or reducing the perception of stress symptoms, in particular in male individuals with moderate hypertension, i.e. with mean systolic pressure values ranging from 125 to 140 mmHg.

In a further aspect thereof, the invention relates to a method for preventing or reducing the perception of stress symptoms, in particular among the male population, and more particularly among male individuals with moderate hypertension, i.e. with mean systolic pressure values ranging from 125 to 140 mmHg, comprising the administration of grape seed extracts.

The grape seed extracts useful for the use or method according to the invention are grape seed extracts characterised by a proanthocyanidin content (typically measured by the Folin UV method) exceeding 95% by weight, and a catechin and epicatechin content ≥5% and ≤15% by weight (typically measured by HPLC).

A grape seed extract commercially available from Indena S.p.A., Italy, under the trademark Enovita™ is preferably used; said extract can be conveniently obtained by extraction under heating with water and subsequent column chromatography on polymer resin.

The extracts can be administered orally at the dose of 150 mg twice a day, preferably before breakfast and before lunch. The extracts can be formulated by techniques known to the skilled person for the preparation of liquid or solid pharmaceutical foul's, using known excipients suitable for said purpose. Said formulations and excipients are described, for example, in Remington, “The Science and Practice of Pharmacy”, 22^{nd} edition, Pharmaceutical Press, 2013. The extracts can preferably be formulated as tablets, each containing 150 mg of extract.

The invention is described in detail in the experimental section below.

Experimental Section

Description of Study

The grape seed extract used was Enovita^{TM} extract, commercially available from Indena S.p.A., Milan (IT), in tablet foimulation, each tablet containing 150 mg of extract.

Table 1 shows the composition of the tablets containing the extract, while Table 2 shows the composition of the placebo tablets used in the study

TABLE 1

| INGREDIENT | mg/tablet |
|---|---|
| Grape seeds, dried extract | 150.0 mg |
| ENOVITA ® | |
| Anhydrous dibasic calcium phosphate | 190.0 mg |
| Di-cafos ® A150 E341(ii) | |
| Microcrystalline cellulose 302 | 147.0 mg |
| Polyvinylpolypyrrolidone | 27.0 mg |
| Polyplasdone ® XL E1202 | |
| Crosslinked sodium | 15.0 mg |
| carboxymethylcellulose | |
| Silicon dioxide | 11.0 mg |
| (Syloid ® 244 FP) | |
| Magnesium stearate | 5.0 mg |
| Talc | 5.0 mg |
| Film-coating agent | 30.0 mg |
| Opadry ® white and purple | |
| Hydroxypropyl methylcellulose | |
| Titanium dioxide | |
| Polydextrose | |
| Talc | |
| Maltodextrin | |
| Medium-chain triglycerides | |
| Cochineal | |

TABLE 2

| INGREDIENT | mg/tablet |
|---|---|
| Anhydrous dibasic calcium phosphate | 245.0 mg |
| Di-cafos ® A150 E341(ii) | |
| Microcrystalline cellulose 302 | 362.0 mg |
| Silicon dioxide | 13.0 mg |
| Syloid ® 244 FP | |
| Magnesium stearate | 15.0 mg |
| Talc | 15.0 mg |
| Film-coating agent | 30.0 mg |
| Opadry ® white and purple | |
| Hydroxypropyl methylcellulose | |
| Titanium dioxide | |
| Polydextrose | |
| Talc | |
| Maltodextrin | |
| Medium-chain triglycerides | |
| Cochineal | |

The double-blind, randomised, placebo-controlled study was conducted on a population of non-smokers consisting of 80 subjects, 35 menopausal women and 45 men, aged between 40 and 70 years, having a mean systolic pressure (measured for a period of seven days) ranging between 125 and 140 mmHg but not receiving any pharmacological treatment, and having a BMI (body mass index) ranging between 19 and 32 kg/m². Individuals with a diet rich in polyphenols, i.e. those with an intake of five or more portions of fruit and vegetables a day, were excluded from the study.

The tablets were administered twice a day, shortly before (5-10 minutes before) breakfast and lunch for a period of sixteen weeks.

The perception of stress symptoms was evaluated with the “Perceived Stress Questionnaire” (PSQ20) (Fliege et al. Psychosom Med. 2005 January-February; 67(1):78-88), a well-established method that evaluates the stress perceived by a subject independently of a specific or objective situation. The test considers four aspects: worry, tension, joy and demands, each with five items. The questionnaire was completed at three different times, on the first day of administration of the grape seed extract (T-1), then 57 (fifty-seven) days (T-2) and 113 (one hundred and thirteen) days (T3) after the start of administration.

Results

The results of PSQ20 are set out in the tables below, each relating to one of the aspects evaluated in the test.

TABLE 3

| | | | | Grape seed | Grape seed | Grape seed |
|---|---|---|---|---|---|---|
| | Placebo tablets | Placebo tablets | Placebo tablets | extract tablets | extract tablets | extract tablets |
| Feeling of worry | T1 | T2 | T3 | T1 | T2 | T3 |
| Number of values | 38 | 38 | 38 | 40 | 40 | 40 |
| Minimum | 0 | 0 | 0 | 0 | 0 | 0 |
| 25th percentile | 0 | 0 | 0 | 6.67 | 0 | 0 |
| Median | 20 | 10 | 20 | 13.33 | 13.33 | 6.67 |
| 75 th percentile | 28.34 | 28.34 | 33.33 | 26.67 | 20 | 20 |
| Maximum | 53.33 | 53.33 | 60 | 60 | 60 | 53.33 |
| Mean | 17.9 | 14.9 | 18.4 | 16.5 | 14.7 | 13.7 |
| Std Deviation | 14.5 | 15.5 | 16.3 | 14.9 | 14.6 | 16.0 |
| Std Error | 2.3 | 2.5 | 2.6 | 2.4 | 2.3 | 2.5 |
| Lower limit of 95% | 13.1 | 9.8 | 13.1 | 11.7 | 10.0 | 8.6 |
| confidence interval | | | | | | |
| Upper limit of 95% | 22.7 | 20.0 | 23.8 | 21.3 | 19.3 | 18.8 |
| confidence interval | | | | | | |

TABLE 4

| | | | | Grape seed | Grape seed | Grape seed |
|---|---|---|---|---|---|---|
| | Placebo tablets | Placebo tablets | Placebo tablets | extract tablets | extract tablets | extract tablets |
| Tension | T1 | T2 | T3 | T1 | T2 | T3 |
| Number of values | 38 | 38 | 38 | 40 | 40 | 40 |
| Minimum | 0 | 0 | 0 | 0 | 0 | 0 |
| 25th percentile | 13.33 | 6.67 | 6.67 | 8.335 | 6.67 | 1.668 |
| Median | 26.67 | 23.34 | 20 | 20 | 20 | 20 |
| 75 th percentile | 35 | 40 | 40 | 33.33 | 33.33 | 33.33 |
| Maximum | 66.67 | 60 | 66.67 | 66.67 | 73.33 | 86.67 |
| Mean | 26.5 | 24.7 | 25.3 | 21.3 | 21.5 | 20.3 |
| Std Deviation | 16.1 | 17.6 | 19.9 | 17.2 | 17.0 | 18.7 |
| Std Error | 2.6 | 2.9 | 3.2 | 2.7 | 2.7 | 3.0 |
| Lower limit of 95% | 21.2 | 19.0 | 18.7 | 15.8 | 16.1 | 14.3 |
| confidence interval | | | | | | |
| Upper limit of 95% | 31.8 | 30.5 | 31.8 | 26.8 | 26.9 | 26.3 |
| confidence interval | | | | | | |

TABLE 5

| | Placebo tablets | Placebo tablets | Placebo tablets | Grape seed extract | Grape seed extract | Grape seed extract |
|---|---|---|---|---|---|---|
| Joy | T1 | T2 | T3 | tablets T1 | tablets T2 | tablets T3 |
| Number of values | 38 | 38 | 38 | 40 | 40 | 40 |
| Minimum | 26.67 | 33.33 | 26.67 | 40 | 26.67 | 33.33 |
| 25th percentile | 65 | 58.33 | 60 | 60 | 61.67 | 60 |
| Median | 73.33 | 73.33 | 73.33 | 73.33 | 73.33 | 73.33 |
| 75 th percentile | 86.67 | 86.67 | 88.34 | 86.67 | 86.67 | 86.67 |
| Maximum | 100 | 100 | 100 | 100 | 100 | 100 |
| Mean | 71.6 | 72.5 | 71.4 | 73.2 | 72.8 | 74.3 |
| Std Deviation | 18.4 | 18.7 | 20.9 | 16.8 | 19.2 | 17.8 |
| Std Error | 3.0 | 3.0 | 3.4 | 2.7 | 3.0 | 2.8 |
| Lower limit of 95% | 65.5 | 66.3 | 64.6 | 67.8 | 66.7 | 68.6 |
| confidence interval | | | | | | |
| Upper limit of 95% | 77.6 | 78.6 | 78.3 | 78.6 | 79.0 | 80.0 |
| confidence interval | | | | | | |

TABLE 6

| | | | | Grape seed | Grape seed | Grape seed |
|---|---|---|---|---|---|---|
| | Placebo tablets | Placebo Tablets | Placebo Tablets | extract tablets | extract tablets | extract tablets |
| Demands | T1 | T2 | T3 | T1 | T2 | T3 |
| Number of values | 38 | 38 | 38 | 40 | 40 | 40 |
| Minimum | 0 | 0 | 0 | 0 | 0 | 0 |
| 25 th percentile | 20 | 13.33 | 20 | 13.33 | 20 | 8.335 |
| Median | 33.33 | 30 | 20 | 30 | 26.67 | 26.67 |
| 75th percentile | 53.33 | 46.67 | 46.67 | 46.67 | 46.67 | 33.33 |
| Maximum | 66.67 | 73.33 | 80 | 66.67 | 80 | 66.67 |
| Mean | 35.1 | 31.4 | 30.7 | 28.3 | 29.5 | 26.5 |
| Std Deviation | 18.6 | 20.2 | 21.8 | 19.1 | 18.1 | 18.8 |
| Std Error | 3.0 | 3.3 | 3.5 | 3.0 | 2.9 | 3.0 |
| Lower limit of 95% | 29.0 | 24.8 | 23.5 | 22.2 | 23.7 | 20.5 |
| confidence interval | | | | | | |
| Upper limit of 95% | 41.2 | 38.0 | 37.9 | 34.4 | 35.3 | 32.5 |
| confidence interval | | | | | | |

The results set out in Tables 3-6 above demonstrate that while the variation in the feeling of joy was not significant (p=0.6314), there was a significant reduction (p=0.00199) in the feeling of worry in the group of subjects treated with grape seed extract, and said significance was also confirmed in the comparison with the placebo; moreover, a moderate reduction in the feeling of tension (p=0.6065) was observed in the treated subjects. It can therefore be concluded that administration of the grape seed extract reduces the perception of stress symptoms, in particular the perception of the feeling of worry.

1. A method of preventing or reducing perception of stress symptoms in subjects in need thereof, said method comprising
administering to said subjects a pharmaceutical effective amount of grape seed extracts.

2. The method according to claim 1, said extracts having a proanthocyanidin content greater than 95% by weight and a catechin and epicatechin content ≥5% and ≤15% by weight.

3. The method according to claim 1, wherein said subjects belong to the male population.

4. A method of preventing or reducing feelings of worry or tension in subjects in need thereof, said method comprising,
administering to said subjects a pharmaceutical effective amounts of grape seed extracts.

5. A method of preventing or reducing feelings of worry in subjects in need thereof, said method comprising,
administering to said subjects a pharmaceutical effective amounts of grape seed extracts.

6. The method according to claim 1, wherein said pharmaceutical effective amount of grape seed extracts comprises 150 mg of extracts twice a day.

7. The method according to claim 1, wherein said pharmaceutical effective amount of grape seed extracts is administered orally.

8. The method according to claim 4, said extracts having a proanthocyanidin content greater than 95% by weight and a catechin and epicatechin content ≥5% and ≤15% by weight.

9. The method according to claim 4, wherein said subjects belong to the male population.

10. The method according to claim 4, wherein said pharmaceutical effective amount of grape seed extracts comprises 150 mg of extracts twice a day.

11. The method according to claim 4, wherein said pharmaceutical effective amount of grape seed extracts is administered orally.
